(19) United States
(12) Patent Application Publication
(10) Pub. No.: US 2022/0344008 A1
(43) Pub. Date: Oct. 27, 2022

(54) METHODS AND SYSTEMS FOR AUTOMATICALLY PREDICTING CLINICAL STUDY OUTCOMES

(71) Applicant: Microsoft Technology Licensing, LLC, Redmond, WA (US)

(72) Inventors: Nut LIMSOPATHAM, Bellevue, WA (US); Liang DU, Redmond, WA (US); Robin ABRAHAM, Redmond, WA (US)

(21) Appl. No.: 17/377,320

(22) Filed: Jul. 15, 2021

Related U.S. Application Data
(60) Provisional application No. 63/179,587, filed on Apr. 26, 2021.

(51) Int. Cl. G16H 10/20; G16H 50/20; G06F 40/10; G06K 9/00

(52) U.S. Cl. CPC G16H 10/20; G16H 50/20; G06F 40/10; G06K 9/00456

(57) ABSTRACT

The methods and systems may improve the development of protocol documents used for clinical trials. The methods and systems may automatically estimate the likelihood of success or failure of executing a protocol document for a clinical study using a machine learning model that leverages several hundred thousand of past protocol documents and the outcomes of the clinical studies. The methods and systems may highlight sections of the protocol document that may increase a likelihood of an unsuccessful execution of the protocol document and may provide one or more recommendations to improve the highlighted sections of the protocol document.

### CROSS-REFERENCE TO RELATED APPLICATIONS

This application claims priority to and the benefit of U.S. Provisional Patent Application No. 63/179,587, filed on Apr. 26, 2021, which is hereby incorporated by reference in its entirety.

### BACKGROUND

A clinical trial or clinical study is an important process in drug development, where the developed medicine is tested in controlled groups. If the test is successful, the medicine will later be released to the market. A protocol document (or clinical study document) is a document providing detailed information explaining how to conduct a clinical study. A clinical study is an expensive process, as the clinical study includes finding and recruiting patients and practitioners, and/or preparing physical labs, medicines, or chemicals for testing with the groups of patients. A failure during the study requires the rewriting of the protocol document and repeating the whole trial process according to the new protocol. Therefore, developing the protocol document is a crucial step for a clinical trial or clinical study.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One example implementation relates a method. The method may include receiving a protocol document for a clinical study for a medical condition. The method may include identifying text from one or more sections of the protocol document. The method may include processing the text from the one or more sections using a plurality of machine learning models, wherein each section of the one or more sections is processed by a different machine learning model of the plurality of machine learning models. The method may include generating an output with a predicted clinical study outcome for the clinical study using the protocol document based on the processing.

Another example implementation relates to a method implemented by a machine learning system to provide recommendations for improving a protocol document for a clinical study for a medical condition. The method may include receiving an output with a predicted clinical study outcome for the clinical study using the protocol document and at least one highlighted section of the protocol document. The method may include accessing one or more datastores with a plurality of historical protocol documents and associated clinical study outcomes. The method may include analyzing the plurality of historical protocol documents and the associated clinical study outcomes. The method may include providing one or more recommendations for the at least one highlighted section based on analyzing the plurality of historical protocol documents and the associated clinical study outcomes.

Another example implementation relates to a system. The system may include a memory to store data and instructions; and at least one processor operable to communicate with the memory, wherein the at least one processor is operable to: receive a protocol document for a clinical study for a medical condition; identify text from one or more sections of the protocol document; process the text from the one or more sections using a plurality of machine learning models, wherein each section of the one or more sections is processed by a different machine learning model of the plurality of machine learning models; and generate an output with a predicted clinical study outcome for the clinical study using the protocol document based on the processing.

Additional features and advantages will be set forth in the description that follows. Features and advantages of the disclosure may be realized and obtained by means of the systems and methods that are particularly pointed out in the appended claims. Features of the present disclosure will become more fully apparent from the following description and appended claims, or may be learned by the practice of the disclosed subject matter as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. Understanding that the drawings depict some example embodiments, the embodiments will be described and explained with additional specificity and detail through the use of the accompanying drawings in which:

FIG. 1 illustrates an example environment in accordance with implementations of the present disclosure.

FIG. 2 illustrates an example machine learning system for use with implementations of the present disclosure.

FIG. 3 illustrates an example self-attention mechanism for use with implementations of the present disclosure.

FIG. 4 illustrates an example graphical user interface (GUI) displaying an output of the machine learning system in accordance with implementations of the present disclosure.

FIGS. 5A and 5B illustrate an example method for predicting an outcome of a clinical study using a protocol document in accordance with implementations of the present disclosure.

### DETAILED DESCRIPTION

A clinical trial or clinical study is an important process in drug development, where the developed medicine is tested in controlled groups. A protocol document (or clinical study document) is a document providing detailed information for how to conduct a clinical study. For example, the protocol documents includes information, such as, study title, study description, study design, steps of the procedure, eligibility criteria, and/or how the results are measured. The protocol document is written before the clinical study is started and individuals are recruited to participate in the clinical study.

Normally, a protocol document is written and reviewed by committee of experts to determine whether the protocol document is in good shape to execute. In many cases, an error occurs in the clinical study based on the protocol document (e.g., a correct number of patients with the given conditions are unable to be recruited for the clinical study). Recent attempts have been made to develop a regressive model that predicts a complexity score that is estimated by experts on different aspects of the protocol document (e.g., chemical used, eligible criteria). However, the recent attempts only predict a complexity of the protocol document.

The present disclosure provides methods and systems for improving the development of the protocol documents used with clinical trials, and thus, reducing the failures of the clinical trials, which may costs multiple million dollars to the pharmaceutical industry. In particular, the present disclosure automatically estimates the likelihood of success and/or failure of executing a protocol document for a clinical study using a statistical machine learning model that leverages a large volume of past protocol documents (e.g., several hundred thousand) and the outcomes of the clinical studies. In addition, to the likelihood of the success and/or failure, the present disclosure also highlights risky parts of the protocol document and provides recommendations for resolving the risky aspects. As such, the present disclosure includes several practical applications that provide benefits and/or solve problems associated with the development of protocol documents.

The present disclosure uses a deep learning system that leverages historical information as a ground truth to train a prediction model to estimate the likelihood of a successful execution of a protocol document without a termination or withdrawal of the clinical trial. The machine learning model may be based on hierarchical transformer networks. The machine learning model takes textual information of different sections of a protocol document as inputs and calculates the likelihood of success for the clinicial study using the protocol document.

In addition, the machine learning model may use a self-attention mechanism that provides potential reasons and/or highlights sections of the protocol document that make the protocol document risky (e.g., that the protocol document is likely to fail resulting in the termination or withdrawal of the clinical study). The machine learning model may provide one or more recommendations to improve the highlighted sections of the protocol document. The recommendations may be extracted from a database of successful clinical studies selected based on a similarity to the protocol input. For example, the recommendations may be based on a clinical study for the same disease or medical condition as the protocol document.

As such, the present disclosure may be used to automatically estimate the likelihood of success and/or failure of a clinical study by executing a protocol document for the clinical study.

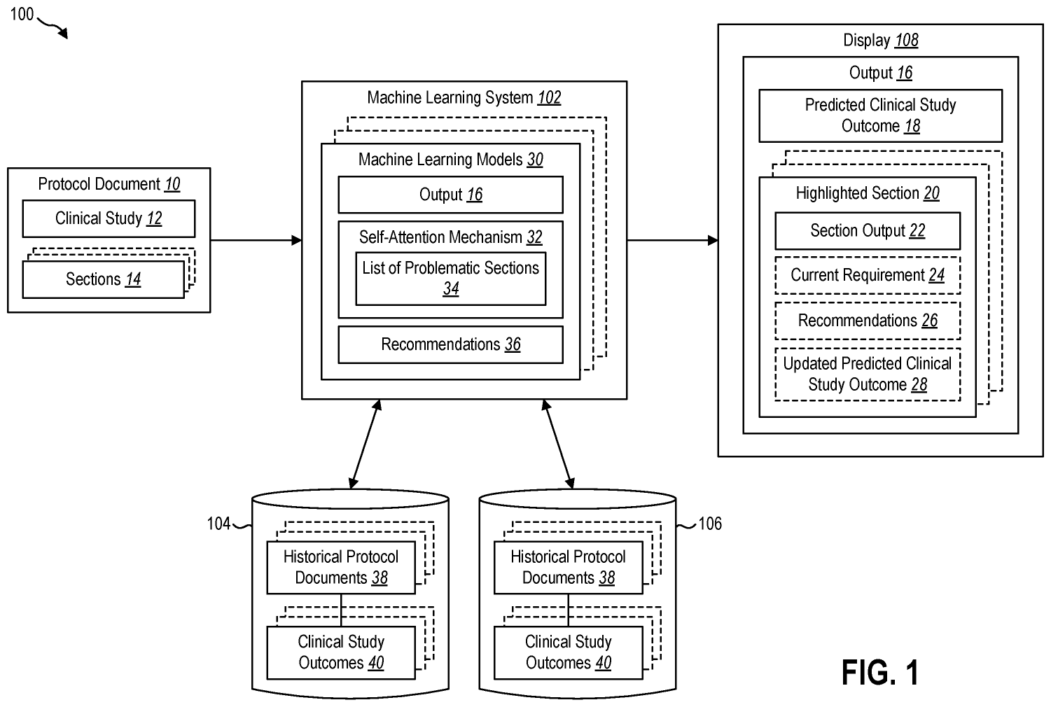

Referring now to FIG. 1, illustrated is an example environment 100 for use with analyzing protocol documents 10 for a clinical study 12. The clinical study 12 is an important process in medicine development, where the developed medicine is tested in controlled groups of individuals or patients (e.g., cohorts of patients). If the clinical study is successful, the developed medicine may be released to the market. A protocol document 10 provides detailed information for conducting the clinical study 12. The protocol documents 10 may include a plurality of sections 14, where each section 14 provides different information for the clinical study 12. For example, the sections 14 may include, but are not limited to, study title, study description, study design, steps of the procedure, eligibility criteria of individuals for the study, and/or how the results are measured for the study.

A protocol document 10 may be provided to a pre-trained machine learning system 102. The pre-trained machine learning system 102 may analyze the text of the protocol document 10 and generate an output 16 based on the analysis of the text. The output 16 may be presented on a display 108. The output 16 may include a predicted clinical study outcome 18 indicating a likelihood of success for the clinical study 12 using the protocol document 10. For example, the predicted clinical study outcome 18 is a percentage indicating the likelihood of success (e.g., a 75% chance that the clinical study 12 will complete).

The machine learning system 102 may be trained using a database of protocol documents used for previous clinical studies as the training input. The protocol documents may cover a variety of medical conditions and/or diseases. In addition, the training input may include several hundred thousand of past protocol documents for previously performed clinical studies. As such, the corpus used for the training input of the machine learning system 102 may include a large volume of existing protocol documents for past clinical studies performed for different medical conditions and/or diseases. The machine learning system 102 may be a self-learning system that identifies trends and/or criteria in the protocol documents used as the training input that resulted in successful and/or unsuccessful clinical studies 12 and uses the identified trends and/or criteria in determining the predicted clinical study outcome 18. A successful clinical study includes clinical studies 12 that run to the end and provide an answer whether the proposed treatment is effective for the given disease or not effective for the given disease. A successful clinical study also includes clinical studies 12 where the drug or proposed treatment has a positive or significant effect on the disease of interest. An unsuccessful clinical study is when the clinical study 12 is withdrawn or terminated (cannot be completed).

The environment 100 may have multiple machine learning systems 102 and/or machine learning models running simultaneously. For example, the machine learning system 102 may use one or more machine learning models 30 for natural language processing (NLP) to analyze the text of the protocol document 10. For example, the machine learning models 30 may be transformer networks, such as, but not limited to Bidirectional Encoder Representations from Transformers (BERT) models and/or Generative Pre-Trained Transformers (GPT). The transformer networks may be trained by processing the raw data of text from the protocol documents. Other examples of the machine learning models may include, but are not limited to, Embeddings from Language Models (ELMO), and/or any other machine learning model for NLP.

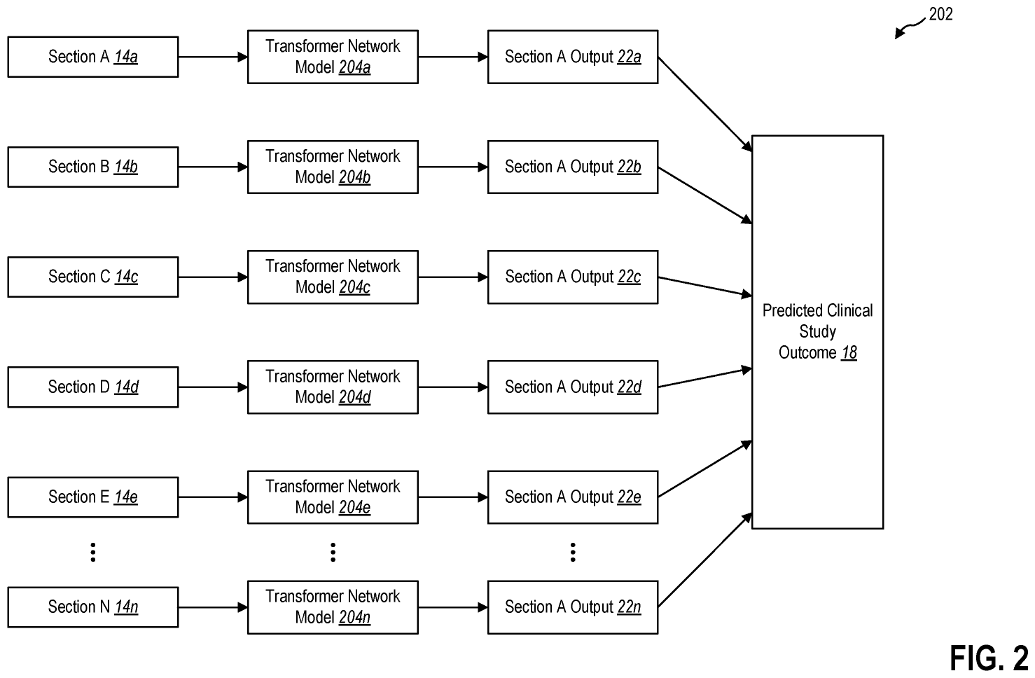

Referring now to FIG. 2, illustrated is an example machine learning system 202 for use with environment 100 to analyze a protocol document 10 (FIG. 1). In some implementations, the machine learning system 202 may be used as part of the machine learning system 102. The machine learning system 202 uses a plurality of transformer network models 204a-n to analyze the different sections 14a-n of the protocol document 10. For example, section A 14a of the protocol document 10 is a study description of the clinical study 12. Section B 14b of the protocol document 10 is a study design explaining the target of the cohorts of patients or individuals for the clinical study 12. Section C 14c is the groups and cohorts for the clinical study 12 (which patients or individuals to include in the clinical study 12). Section D 14d is the outcome measures for the clinical study 12 (which outcomes of the clinical study 12 to measure for success). Section E 14e is the eligibility criteria of the patients or individuals for the clinical study 12 (e.g., genders, age ranges, medical conditions to include, and/or medical conditions to exclude).

While five sections of the protocol document are illustrated, any number of sections up to n (where n is a positive integer) may be included in the protocol document 10. Moreover, while five transformer network models are illustrated any number of transformer network models up to n (where n is a positive integer) may be used by machine learning system 202. In addition, the number of transformer network models 204a-n used by machine learning system 202 may equal the number of sections 14a-n included in the protocol document 10. As such, a different transformer network model 204a-n may be used for each section 14a-n of the protocol document 10.

For example, section A 14a of the protocol document 10 is analyzed by the transformer network model 204a. The transformer network model 204a generates a section A output 22a indicating a likelihood of success for implementing section A 14a during the clinical study 12. The transformer network model 204b analyzes section B 14b of the protocol document 10 and generates a section B output 22b. The section B output 22b indicates a likelihood of success for implementing section B 14b of the protocol document 10 during the clinical study 12. The transformer network model 204c analyzes section C 14c of the protocol document 10 and generates a section C output 22c. The section C output 22c indicates a likelihood of success for implementing section C 14c of the protocol document 10 during the clinical study 12.

The transformer network model 204d analyzes section D 14d of the protocol document 10 and generates a section D output 22d. The section D output 22d indicates a likelihood of success for implementing section D 14d if the protocol document 10 during the clinical study 12. The transformer network model 204e analyzes section E 14e of the protocol document 10 and generates a section E output 22e. The section E output 22e indicates a likelihood of success for implementing section E 14e if the protocol document 10 during the clinical study 12.

The machine learning system 202 may take the average of the section outputs (e.g., section A output 22a, section B output 22b, section C output 22c, section D output 22d, section E output 22e) in calculating the predicted study outcome 18. In addition, different ranks and/or weights may be applied to the different section outputs 22a-n when calculating the predicted clinical study outcome 18. For example, the machine learning system 202 may place a higher weight on Section C 14c (which patients to include in the clinical study 12) and Section E 14e (eligibility criteria of the patients) relative to a weight of Section A 14a (study description). As such, the machine learning system 202 may use different weights on different sections 14 of the protocol document 10 in calculating the predicted study outcome 18.

The section outputs (e.g., section A output 22a, section B output 22b, section C output 22c, section D output 22d, section E output 22e) may be in a variety of forms. For example, the section outputs 22a-n may be a percentage indicating the likelihood of success (e.g., 60% chance of success) and the machine learning model may use the percentages in calculating the predicted study outcome 18. In another example, the section outputs 22a-n may be a color indicating the likelihood of success where green is likely to be successful, yellow might be unsuccessful, and red is likely to be unsuccessful. The machine learning system 202 may use the different colors in determining the predicted study outcome 18. In another example, the section outputs 22a-n may be scores indicating the likelihood of success and the machine learning system 202 may use the scores in determining the predicted study outcome 18.

Referring back to FIG. 1, the machine learning system 102 may also use one or more machine learning models 30 to provide recommendations 26 for improving the predicted clinical study outcome 18. The output 16 may also include one or more highlighted sections 20 of the protocol document 10 that may cause a risk of the clinical study 12 failing or increase a likelihood of the clinical study 12 failing. The highlighted sections 20 may correspond to one or more sections 14 of the protocol document where the section output 22 is lower than a threshold value. For example, the threshold value may be 90% and the highlighted sections 20 include any section output 22 that is lower than 90%. Another example may include the threshold value with a green color and the highlighted sections 20 include any section output 22 that is a color other than green (e.g., red or yellow).

The output 16 may also include the current requirements 24 for the highlighted sections 20. The current requirements 24 may include text or other information from the corresponding sections 14 of the protocol document 10. For example, the highlighted section 20 may be the eligibility section 14 and the current requirements 24 may include patients age between 40 years old and 80 years old.

The machine learning system 102 may use the machine learning models 30 to verify the risk for the highlighted sections 20. The machine learning models 30 may use a self-attention mechanism 32 to determine a list of problematic sections 34 in the protocol document 10. The list of problematic sections 34 may correspond to the highlighted sections 20. The list of problematic sections 34 may identify the sections 14 and/or the current requirements 24 of the sections in the protocol document 10 that may result in a termination of the clinical study 12 prior to completion of the clinical study 12. The list of problematic sections 34 may also identify the sections 14 and/or the current requirements 24 of the sections in the protocol document 10 that may result in an unsuccessful clinical study 12. An unsuccessful clinical study 12 is when the study must be withdrawn or terminated (cannot be completed). Hence, all the money is spent on the clinical study 12 without getting any conclusion for the proposed treatment. Withdrawal or termination of the clinical studies 12 may occur, for example, when the clinical study 12 cannot recruit enough patients or practitioners, and/or the method for measuring the outcome in the clinical study 12 is too difficult or not realistically applicable. The list of problematic sections 34 may identify the sections 14 and/or the current requirements 24 of the sections in the protocol document 10 that may prevent the clinical study 12 from starting. As such, the list of problematic sections 34 may identify sections 14 and/or features of the sections 14 that increase a risk that the protocol document 10 will be unsuccessfully executed by the clinical study 12 resulting in a termination of the clinical study 12 before completion of the clinical study 12 or an unsuccessful clinical study 12.

A ranking may be applied to the list of problematic sections 34 to place the sections 14 and/or the problematic criteria in an order so that the sections 14 and/or problematic criteria with a lower percentage of completion is placed higher in the list relative to the sections 14 and/or the problematic criteria with a higher percentage of completion. The list of problematic sections 34 may be used to provide potential reasons for the unsuccessful execution of the protocol document 10.

The machine learning models 30 may use the list of problematic sections 34 to identify one or more recommendations 26 to improve the likelihood of success for the identified sections 14 and/or the problematic criteria and retrieve potential improvements for the identified sections 14 and/or the problematic criteria.

The machine learning models 30 may access one or more datastores 104, 106 up to m datastores (where m is a positive integer) for the potential improvements. The datastores 104, 106 may include a plurality of historical protocol documents 38 and associated clinical study outcomes 40 from clinical studies 12 that have previously occurred. The datastores 104, 106 may store the plurality of historical protocol documents 38 by medical condition and/or disease. In addition, the datastores 104, 106 may be populated with historical protocol documents 38 and clinical study outcomes 40 by different content providers. For example, the datastore 104 may include clinical study outcomes 40 from a pharmaceutical company and the datastore 106 may include clinical study outcomes 40 from a different pharmaceutical company. In another example, the datastore 104 may include clinical study outcomes 40 from a government research group and the datastore 106 may include clinical study outcomes 40 from a university research group.

The machine learning models 30 may be applied to the clinical study outcomes 40 to identify successful clinical study outcomes 40 and the associated historical protocol documents 38. In addition, the machine learning models 30 may be applied to the clinical study outcomes 40 to identify unsuccessful clinical study outcomes 40 and the associated historical protocol documents 38. The machine learning models 30 may identify trends and/or criteria in the historical protocol documents 38 that resulted in successful and/or unsuccessful clinical study outcomes 40.

The machine learning models 30 may retrieve potential improvements to the list of problematic sections 34 based on a similarity of the protocol document 10 and one or more clinical studies in the datastores 104, 106. In some implementations, the machine learning models 30 may identify one or more related clinical studies in the datastores 104, 106 to use in identifying potential improvements to the protocol document 10. Examples of related clinical studies may include, but are not limited to, clinical studies for the same or similar diseases or conditions as included in the protocol document 10, clinical studies for the same or similar cohort of patients as included in the protocol document 10, clinical studies that use the same or similar output measures as included in the protocol document 10, and/or clinical studies with the same or similar study description as included in the protocol document. The machine learning models 30 may use the clinical study outcomes 40 for the related clinical studies to identify one or more recommendations 26 for improving the likelihood of success for the highlighted sections 20 of the protocol document 10.

The recommendations 26 may be based on information learned from the successful and/or unsuccessful clinical study outcomes 40. For example, the successful clinical study outcomes 40 may be used to identify different criteria of success for the clinical study 12. The different criteria of success may be identified from different requirements of the historical protocol documents 38 used for the successful clinical study outcomes 40. One or more recommendations 26 may be provided based on the identified criteria of success for the related clinical studies.

The output 16 of the machine learning system 102 may also include one or more recommendations 26 for improving the highlighted sections 20 and an updated predicted clinical study outcome 28 based on implementing the one or more recommendations 26. For example, the recommendations 26 may include changing the age requirement for patients in the eligibility section 14 from patients with an age between 40 years old and 80 years old to patients with an age between 20 years old and 80 years old and the updated predicted clinical study outcome 28 may change from 70% to 85% if this recommendation 26 is implemented for the eligibility section 14.

The output 16 may be presented on a display 108. The display 108 may be on a computing device in communication with the machine learning system 102. In some implementations, a user of the computing device may use the output 16 to revise and/or update the protocol document 10 based on the predicted clinical study outcome 18, the highlighted sections 20, and/or any recommendations 26. In some implementations, the computing device may use the output 16 to automatically revise and/or update the protocol document 10 based on the predicted clinical study outcome 18, the highlighted sections 20, and/or any recommendations 26. Any updates and/or changes to the protocol document 10 may be visually distinct from the original protocol document 10 so a user can easily identify the changes made to the protocol document 10. The user may approve or reject the changes to the protocol document 10.

In some implementations, one or more computing devices (e.g., servers and/or devices) are used to perform the processing of environment 100. The one or more computing devices may include, but are not limited to, server devices, personal computers, a mobile device, such as, a mobile telephone, a smartphone, a PDA, a tablet, or a laptop, and/or a non-mobile device. The features and functionalities discussed herein in connection with the various systems may be implemented on one computing device or across multiple computing devices. For example, the machine learning system 102, the datastores 104, 106, and/or the display 108 are implemented wholly on the same computing device. Another example includes one or more subcomponents of the machine learning system 102, the datastores 104, 106, and/or the display 108 implemented across multiple computing devices. Moreover, in some implementations, the machine learning system 102, the datastores 104, 106, and/or the display 108 may be implemented are processed on different server devices of the same or different cloud computing networks.

In some implementations, each of the components of the environment 100 is in communication with each other using any suitable communication technologies. In addition, while the components of the environment 100 are shown to be separate, any of the components or subcomponents may be combined into fewer components, such as into a single component, or divided into more components as may serve a particular embodiment. In some implementations, the components of the environment 100 include hardware, software, or both. For example, the components of the environment 100 may include one or more instructions stored on a computer-readable storage medium and executable by processors of one or more computing devices. When executed by the one or more processors, the computer-executable instructions of one or more computing devices can perform one or more methods described herein. In some implementations, the components of the environment 100 include hardware, such as a special purpose processing device to perform a certain function or group of functions. In some implementations, the components of the environment 100 include a combination of computer-executable instructions and hardware.

As such, environment 100 may be used to automatically estimate the likelihood of success and/or failure of executing a protocol document 10 for a clinical study 12 using the machine learning system 102. In addition, environment 100 may be used to reduce the failures of clinical studies by improving the development of the protocol document 10 by identify sections 14 of the protocol document 10 that may provide a risk of the clinical study 12 terminating or failing (e.g., fail to have the tested drug or medicine go to market) and providing recommendations 26 to improve the likelihood of success for the clinical study 12.

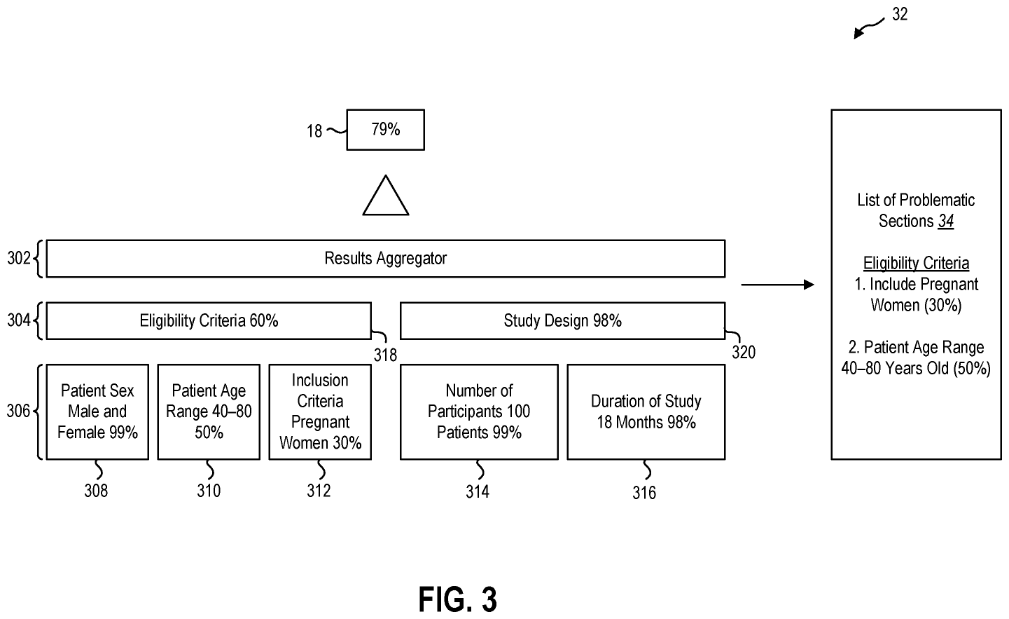

Referring now to FIG. 3, illustrated is an example self-attention mechanism 32 for use with environment 100. The self-attention mechanism 32 may be executed by one or more machine learning models 30 (FIG. 1) of the machine learning system 102 (FIG. 1). The machine learning models 30 may evaluate the different sections 14 of the protocol document 10 (FIG. 1) and the corresponding section outputs 22 to identify one or more sections 14 that may include problematic criteria that may prevent the successful execution of the protocol document 10. The machine learning models 30 may further evaluate the sections 14 to identify which criteria may be problematic resulting in an unsuccessful execution of the protocol document 10, and thus, having the clinical study 12 terminate or fail (e.g., fail to have the tested drug or medicine go to market).

In the illustrated example, a section layer 304 with two sections 14 may include the eligibility criteria 318 and the study design 320. The text layer 306 illustrates the text (e.g., words, phrases, bullet points, and/or sentences) from the protocol document 10 for each section 14. The eligibility criteria 318 for the patients to include in the clinical study 12 may include, for example, the patient sex 308 (e.g., male and female), the patient age range 310 (e.g., 40-80 years old), and inclusion criteria 312 (e.g., include pregnant women). The study design 320 for the clinical study 12 may include, for example, a number of participants (e.g., 100 participants) and the duration of the study 316 (e.g., 18 months). The machine learning models 30 may analyze the text in the text layer 306 to identify the different sections 14 of the protocol document 10 and the requirements of the different sections 14.

In addition, the machine learning models 30 may analyze the text layer 306 and determine a predicted outcome of the clinical study 12 based on the requirements of the different sections 14. For example, for the eligibility criteria 318 section, the machine learning models 30 may predict that the patient sex 308 criteria has a 99% chance of completion by the clinical study 12 (e.g., the clinical study 12 has a high likelihood of finding the correct patient sex for the clinical study 12). The machine learning models 30 may predict that the patient age range 310 has a 50% chance of completion by the clinical study 12 (e.g., the clinical study 12 has about a 50% chance of recruiting patients in the correct age range for the clinical study 12). The machine learning models 30 may predict that the inclusion criteria 312 has a 30% chance of completion by the clinical study 12 (e.g., the clinical study 12 has a low likelihood of recruiting patients with the inclusion criteria 312).

In addition, for the study design 320 section, the machine learning models 30 may predict that the number of participants 314 has a 99% chance of completion by the clinical study (e.g., the clinical study 12 has a high likelihood of recruiting the correct number of patients for the clinical study 12). The machine learning models 30 may predict that the duration of the study 316 has a 98% chance of completion by the clinical study 12.

The machine learning models 30 may generate a predicted outcome for the eligibility criteria 318 section and the study design 320 section based on the different predictions for each portion of text in the text layer 306. For example, the overall predicted outcome for the eligibility criteria 318 section may be 60% (e.g., that the clinical study 12 has a 60% chance of recruiting the patients that meet the eligibility criteria 318 for the clinical study 12). In addition, the overall predicted outcome for the study design 320 section may be 98% (e.g., that the clinical study 12 has a high likelihood of success using the study design).

While only two sections 14 (e.g., eligibility criteria 318 and study design 320) are illustrated, any number of sections up to n (where n is a positive integer) may be included in the disclosure document 10. In addition, any number of text portions up to m may be included in the text section layer 306.

The machine learning models 30 may include a result aggregator layer 302 that aggregates the different predicted outcomes for the sections 14 and generates an overall predicted clinical study outcome 18 for the clinical study 12. For example, the predicted clinical study outcome 18 may be 79% an average of the predicted outcome for the eligibility criteria 318 (e.g., 60%) and the predicted outcome for the study design 320 (98%). The result aggregator layer 302 may apply weights to the predicted outcomes of the different sections and may use the weights in determining the overall predicted clinical study outcome 18. For example, the eligibility criteria 318 may have a higher weight relative to the study design 320. As such, the overall predicted clinical study outcome 18 may be based on an aggregation of the section outputs generated by each machine learning model for each section.

In addition, the machine learning models 30 may generate a list of problematic sections 34 for the different sections 14 of the protocol document 10. The list of problematic sections 34 may include any predicted outcome from the text layer 306 under a threshold level, such as, but not limited to 90%. The list of problematic sections 34 may also be ranked in an ascending order with a lowest predicted outcome (e.g., the lowest likelihood of success) placed first in the list and a higher predicted outcome (e.g., a higher likelihood of success) placed lower in the list. For example, the list of problematic sections 34 includes “Eligibility Criteria Section” as a header for the list with the inclusion criteria listed first along with the predicted outcome (e.g., Include Pregnant Women, Likelihood of Completion 30%) and the patients age range listed second along with the predicted outcome (e.g., Patient Age Range 40-80 years old, Likelihood of Completion 50%).

The list of problematic sections 34 may be used by the machine learning system 102 to identify problematic areas in the protocol document 10 that may prevent a successful clinical study 12. In addition, the list of problematic sections 34 may be used by the machine learning system 102 to provide one or more recommendations for improving the problematic areas of the protocol document 10.

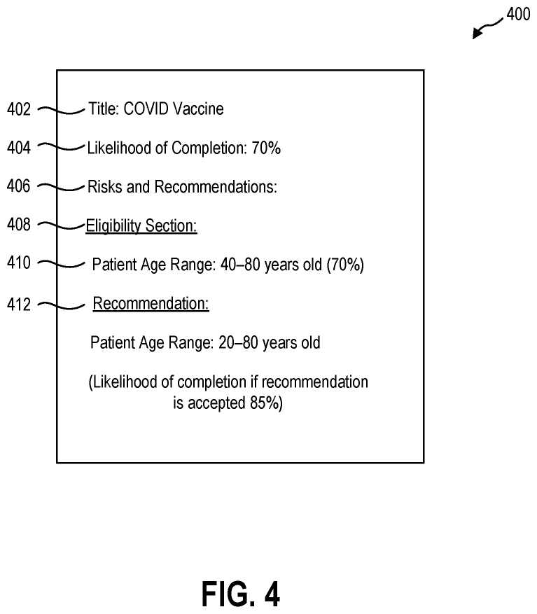

Referring now to FIG. 4, illustrated is an example graphical user interface (GUI) 400 for displaying the output 16 of the machine learning system 102 (FIG. 1) based on the analysis of the protocol document 10 (FIG. 1). The GUI 400 may be displayed on the display 108 of a device in communication with the machine learning system 102.

The output 16 may include the title 402 of the clinical study 12 (e.g., Coronavirus disease (COVID) vaccine) that the protocol document 10 is describing and the likelihood of completion 404 (e.g., 70%) of the clinical study 12 (e.g., the predicted clinical study outcome 18 (FIG. 1)) based on the protocol document 10. The output 16 may also include the identified risks and recommendations 406 of the protocol document 10.

In addition, the output 16 may include a header 408 with the eligibility section listed. The sections included in the header 408 may correspond to the highlighted sections 20 (FIG. 1) that the machine learning system 102 identified that may cause a risk of the clinical study 12 failing. The output 16 may also include the patients age range 410 (e.g., patients between the age of 40 and 80 years) and the predicted likelihood of completion (e.g., the section output 22 (FIG. 1)) of the clinical study 12 based on the patients age range 410 (e.g., 70%). The output 16 may also include one or more recommendations 412 (e.g., the recommendations 36 (FIG. 1) for improving the likelihood of completion of the clinical study 12 and an updated predicted likelihood of completion if the recommendation is accepted (e.g., patient age range 20-80 and likelihood of completion if recommendation is accepted 85%).

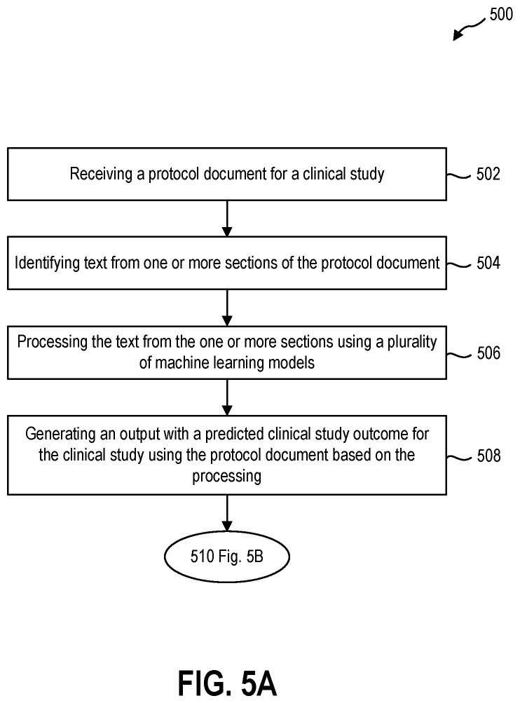

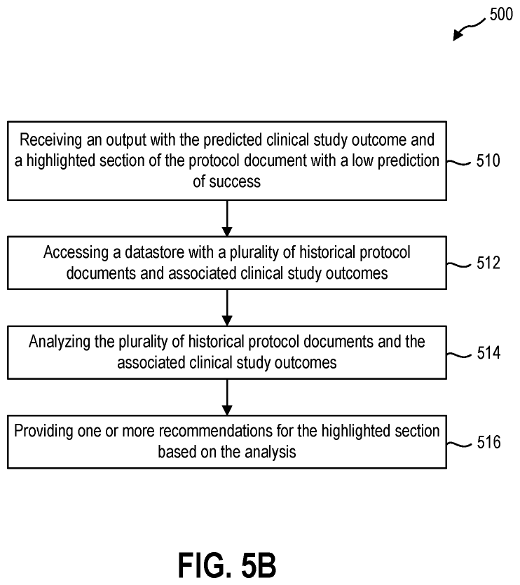

Referring now to FIGS. 5A and 5B, illustrated is an example method 500 for use with environment 100 (FIG. 1) for predicting an outcome of a clinical study 12 (FIG. 1) using a protocol document 10 (FIG. 1). The actions of method 500 may be performed by the machine learning system 102 (FIG. 1) and/or one or more machine learning models 30 (FIG. 1) of the machine learning system 102. The actions of method 500 may be discussed below with reference to the architectures of FIGS. 1-3.

At 502, method 500 includes receiving a protocol document for a clinical study. The protocol document 10 provides detailed information for conducting a clinical study 12 for a new drug or medicine for treating a medical condition or disease. The developed medicine or drug is tested in controlled groups of individuals or patients during the clinical study 12. If the clinical study 12 is successful in testing the new drug or medicine, the new drug or medicine may be provided to the public. The protocol documents 10 may include a plurality of sections 14, where each section 14 provides different information for the clinical study 12. For example, the sections 14 may include, but are not limited to, study title, study description, study design, steps of the procedure, eligibility criteria of individuals for the study, and/or how the results are measured for the study. The protocol document 10 may be provided to a pre-trained machine learning system 102.

At 504, method 500 includes identifying text from one or more sections of the protocol document. The machine learning system 102 may identify the text from one or more sections 14 of the protocol document 10. The text may include, but is not limited to, sentences, phrases, words, and/or bullet points.

At 506, method 500 includes processing the text from the one or more sections using a plurality of machine learning models. The machine learning system 102 may use one or more pre-trained machine learning models 30 for natural language processing (NLP) to analyze the text of the protocol document 10. For example, the machine learning models 30 may be transformer networks, such as, but not limited to Bidirectional Encoder Representations from Transformers (BERT) models and/or Generative Pre-Trained Transformers (GPT).

In an implementation, the machine learning system 102 may use a plurality of transformer network models (e.g., transformer network models 204a-n) to analyze the different sections (e.g., sections 14a-n) of the protocol document 10. The number of transformer network models used by the machine learning system 102 may equal the number of sections included in the protocol document 10. In addition, a different transformer network model may be used for each section of the protocol document 10. As such, each section 14 of the protocol document 10 may be processed by different machine learning models.

At 508, method 500 includes generating an output with a predicted clinical study outcome for the clinical study using the protocol document based on the processing. The pre-trained machine learning system 102 may analyze the text of the protocol document 10 and generate an output 16 based on the analysis of the text. The output 16 may be presented on a display 108. The output 16 may include a predicted clinical study outcome 18 indicating a likelihood of success for the clinical study 12 using the protocol document 10. For example, the predicted clinical study outcome 18 is a percentage indicating the likelihood of success (e.g., a 75% chance that the clinical study 12 will be successful).

At 510, method 500 may include receiving an output with the predicted clinical study outcome and a highlighted section of the protocol document with a low prediction of success. The output 16 may also include one or more highlighted sections 20 of the protocol document 10 that may cause a risk of the clinical study 12 failing (e.g., have a higher likelihood of causing the clinical study 12 to fail). The highlighted sections 20 may correspond to one or more sections 14 of the protocol document where the section output 22 is lower than a threshold value. The output 16 may also include the current requirements 24 for the highlighted sections 20. The current requirements 24 may include text or other information from the corresponding sections 14 of the protocol document 10.

The machine learning system 102 may use the machine learning models 30 to verify the risk for the highlighted sections 20. For example, the machine learning models 30 may use a self-attention mechanism 32 to determine a list of problematic sections 34 in the protocol document 10. The list of problematic sections 34 may correspond to the highlighted sections 20. The list of problematic sections 34 may identify the sections 14 and/or the current requirements 24 of the sections 14 in the protocol document 10 that may result in a termination of the clinical study 12 prior to completion of the clinical study 12 or may cause the clinical study 12 to fail. As such, the list of problematic sections 34 may identify the sections 14 and/or the features of the sections 14 that increase a risk of an unsuccessful execution of the protocol document 10 by the clinical study 12 and that the clinical study 12 will terminate before completion of the clinical study 12 and/or fail.

A ranking may be applied to the list of problematic sections 34 to place the problematic sections and/or criteria in an order so that the problematic sections and/or criteria with a lower percentage of completion is placed higher in the list relative to the problematic sections and/or criteria with a higher percentage of completion. The list of problematic sections 34 may be used to provide potential reasons for the unsuccessful execution of the protocol document 10.

At 512, method 500 may include accessing a datastore with a plurality of historical protocol documents and associated clinical study outcomes. The machine learning models 30 may access one or more datastores 104, 106. The datastores 104, 106 may include a plurality of historical protocol documents 38 and associated clinical study outcomes 40 from clinical studies 12 that have previously occurred.

At 514, method 500 may include analyzing the plurality of historical protocol documents and the associated clinical study outcomes. The machine learning models 30 may analyze the plurality of historical protocol documents 38 and the associated clinical study outcomes 40. For example, the machine learning models 30 may identify successful clinical study outcomes 40 and the associated historical protocol documents 38. The machine learning models may also identify unsuccessful clinical study outcomes 40 and the associated historical protocol documents 38. In addition, the machine learning models 30 may identify trends and/or criteria in the historical protocol documents 38 that resulted in successful and/or unsuccessful clinical study outcomes 40. The machine learning models 30 may retrieve potential improvements to the list of problematic sections 34 based on a similarity of the protocol document 10 and one or more clinical studies in the datastores 104, 106. The machine learning models 30 may use the clinical study outcomes 40 for the clinical studies to identify one or more recommendations 26 for improving the likelihood of success for the highlighted sections 20 of the protocol document 10. The recommendations 26 may be based on the identified trends and/or criteria from the historical protocol documents 38 associated with the successful clinical studies outcomes 40.

At 516, method 500 may include providing one or more recommendations for the highlighted section based on the analysis. The machine learning models 30 may provide recommendations 26 for improving the predicted clinical study outcome 18 based on the analysis of the historical protocol documents 38 and the associated clinical study outcomes 40. The recommendations 26 may be based on information learned from the successful and/or unsuccessful clinical study outcomes 40. The successful clinical study outcomes 40 may be used to identify different criteria of success for the clinical study 12. The different criteria of success may be identified from different requirements of the historical protocol documents 38 used for the successful clinical study outcomes 40. One or more recommendations 26 may be provided based on the identified criteria of success for the related clinical studies. The output 16 of the machine learning system 102 may also include one or more recommendations 26 for improving the highlighted sections 20 and an updated predicted clinical study outcome 28 based on the one or more recommendations 26. The updated predicted clinical study outcome 28 may include a higher prediction of success for the clinical study 12 relative to the original predicted clinical study outcome 18.

As such, method 500 may be used by the machine learning system to automatically estimate the likelihood of success and/or failure of executing a protocol document 10 for a clinical study 12. In addition, method 500 may be used to provide recommendations for improving the protocol document 10.

As illustrated in the foregoing discussion, the present disclosure utilizes a variety of terms to describe features and advantages of the model evaluation system. Additional detail is now provided regarding the meaning of such terms. For example, as used herein, a “machine learning model” refers to a computer algorithm or model (e.g., a transformer model, a classification model, a regression model, a language model, an object detection model) that can be tuned (e.g., trained) based on training input to approximate unknown functions. For example, a machine learning model may refer to a neural network (e.g., a transformer neural network, a convolutional neural network (CNN), deep neural network (DNN), recurrent neural network (RNN)), or other machine learning algorithm or architecture that learns and approximates complex functions and generates outputs based on a plurality of inputs provided to the machine learning model. As used herein, a “machine learning system” may refer to one or multiple machine learning models that cooperatively generate one or more outputs based on corresponding inputs. For example, a machine learning system may refer to any system architecture having multiple discrete machine learning components that consider different kinds of information or inputs.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable storage medium comprising instructions that, when executed by at least one processor, perform one or more of the methods described herein. The instructions may be organized into routines, programs, objects, components, data structures, etc., which may perform particular tasks and/or implement particular data types, and which may be combined or distributed as desired in various embodiments.

Computer-readable mediums may be any available media that can be accessed by a general purpose or special purpose computer system. Computer-readable mediums that store computer-executable instructions are non-transitory computer-readable storage media (devices). Computer-readable mediums that carry computer-executable instructions are transmission media. Thus, by way of example, and not limitation, embodiments of the disclosure can comprise at least two distinctly different kinds of computer-readable mediums: non-transitory computer-readable storage media (devices) and transmission media.

As used herein, non-transitory computer-readable storage mediums (devices) may include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

The steps and/or actions of the methods described herein may be interchanged with one another without departing from the scope of the claims. Unless a specific order of steps or actions is required for proper operation of the method that is being described, the order and/or use of specific steps and/or actions may be modified without departing from the scope of the claims.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one implementation” or “an implementation” of the present disclosure are not intended to be interpreted as excluding the existence of additional implementations that also incorporate the recited features. For example, any element described in relation to an implementation herein may be combinable with any element of any other implementation described herein. Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to implementations disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the implementations that falls within the meaning and scope of the claims is to be embraced by the claims.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method, comprising:
receiving a protocol document for a clinical study for a medical condition;
identifying text from one or more sections of the protocol document;
processing the text from the one or more sections using a plurality of machine learning models, wherein each section of the one or more sections is processed by a different machine learning model of the plurality of machine learning models; and
generating an output with a predicted clinical study outcome for the clinical study using the protocol document based on the processing.

2. The method of claim 1, wherein the protocol document provides detailed information for conducting the clinical study and each section of the protocol document provides different information for the clinical study.

3. The method of claim 1, wherein the plurality of machine learning models are transformer networks.

4. The method of claim 1, wherein the predicted clinical study outcome is based on an aggregation of section outputs for each section of the protocol document generated by the plurality of machine learning models.

5. The method of claim 1, wherein the predicted clinical study outcome includes a percentage indicating a prediction of success of the clinical study using the protocol document.

6. The method of claim 5, wherein the percentage is based on an aggregation of percentages indicating a prediction of success for each section of the protocol document generated by the plurality of machine learning models.

7. The method of claim 5, wherein the output further includes at least one highlighted section of the protocol document with a section output lower than a threshold level indicating a low prediction of success of the clinical study.

8. The method of claim 7, wherein the highlighted section of the protocol document includes a current requirement and the section output.

9. The method of claim 8, wherein the output further includes at least one recommendation for improving the current requirement for the at least one highlighted section and an updated predicted clinical study outcome based on implementing the at least one recommendation.

10. The method of claim 1, wherein the medical condition is a disease and the clinical study is for a drug or medicine for treating the disease.

11. The method of claim 1, wherein the output is presented on a display.

12. A method implemented by a machine learning system to provide recommendations for improving a protocol document for a clinical study for a medical condition, comprising:
receiving an output with a predicted clinical study outcome for the clinical study using the protocol document and at least one highlighted section of the protocol document;
accessing one or more datastores with a plurality of historical protocol documents and associated clinical study outcomes;
analyzing the plurality of historical protocol documents and the associated clinical study outcomes; and
providing one or more recommendations for the at least one highlighted section based on analyzing the plurality of historical protocol documents and the associated clinical study outcomes.

13. The method of claim 12, further comprising:
applying a self-attention mechanism to the protocol document;
generating a list of problematic sections for the protocol document based on the self-attention mechanism.

14. The method of claim 13, wherein the list of problematic sections identifies one or more sections or features of the one or more sections of the protocol document that increase a likelihood of an unsuccessful execution of the protocol document during the clinical study.

15. The method of claim 13, wherein the list of problematic sections is ranked in an ascending order with a problematic section or a criteria with a lowest predicted outcome of successful execution of the protocol document placed first.

16. The method of claim 12, wherein analyzing the plurality of historical protocol documents and the associated clinical study outcomes further includes:
identifying one or more historical protocol documents of the plurality of historical protocol documents that are similar to the protocol document; and
using the one or more historical protocol documents to identify the one or more recommendations.

17. The method of claim 12, wherein analyzing the plurality of historical protocol documents and the associated clinical study outcomes further includes:
identifying successful clinical study outcomes; and
using the historical protocol documents from the successful clinical study outcomes to identify one or more recommendations.

18. The method of claim 17, wherein the one or more recommendations are identified from different requirements of the historical protocol documents associated with the successful clinical study outcomes.

19. The method of claim 12, wherein the at least one highlighted section has a section output lower than a threshold level indicating a low prediction of success of the clinical study, and
wherein the one or more recommendations also include an updated predicted clinical study output indicating a higher prediction of success for the clinical study based on implementing the one or more recommendations.

20. A system comprising,
a memory to store data and instructions; and
at least one processor operable to communicate with the memory, wherein the at least one processor is operable to:
receive a protocol document for a clinical study for a medical condition;
identify text from one or more sections of the protocol document;
process the text from the one or more sections using a plurality of machine learning models, wherein each section of the one or more sections is processed by a different machine learning model of the plurality of machine learning models; and
generate an output with a predicted clinical study outcome for the clinical study using the protocol document based on the processing.
